(19) United States
(12) Patent Application Publication
(10) Pub. No.: US 2008/0182002 A1
(43) Pub. Date: Jul. 31, 2008

(54) Processes for Removing Bitter Components from Soy Protein Isolates

(75) Inventors: Daniel U. Staerk, Kirkwood, MO (US); Parthasarathi Ghosh, Chesterfield, MO (US)

Correspondence Address: Solae, LLC, 4300 Duncan Avenue, Legal Department E4, St. Louis, MO, 63110, US

(73) Assignee: Solae, LLC, St. Louis, MO (US)

(21) Appl. No.: 11/627,664

(22) Filed: Jan. 26, 2007

(51) Int. Cl. A23J 1/14; A23L 1/317; A23L 1/39; A23C 23/00; A23L 2/66; C12P 21/06; C07K 14/415; A23L 1/10; A21D 13/00; A23L 1/31

(52) U.S. Cl. 426/549; 426/641; 426/646; 426/656; 426/589; 426/580; 426/619; 426/598; 530/378; 435/68.1

(57) ABSTRACT

Novel processes for the production of soy protein isolates having reduced off-flavors from conventional hydrolyzed soy protein isolates are disclosed. One embodiment includes an extraction and separation process for removing bitter components to achieve soy protein isolates with reduced bitter flavor. The produced soy protein isolates are suitable for use in a number of food products.

### BACKGROUND OF THE DISCLOSURE

The present disclosure generally relates to processes for removing the bitter components found in conventional hydrolyzed soy protein isolates. In particular, the present disclosure relates to extraction and separation processes for removing bitter components to achieve soy protein isolates with reduced bitter flavor. The produced soy protein isolates are suitable for use in a number of food products.

In response to the results of recent research showing the negative effects of certain foods on health and nutrition, consumers are becoming more health conscious and monitoring their food intake more carefully. In particular, since animal products are the main dietary source of cholesterol and may contain high levels of saturated fats, health professionals have recommended that consumers significantly reduce their intake of red meats. As a substitute, many consumers are choosing soy products.

It is well known that vegetable products, such as soy protein products, contain no cholesterol. For decades, nutritional studies have indicated that the inclusion of soy protein in the diet actually reduces serum cholesterol levels in people who are at risk. Further, the higher the cholesterol level, the more effective soy proteins are in lowering that level.

Despite all of the above advantages, it is well known that by supplementing foods with increased levels of dietary fiber and protein, taste can be seriously compromised. More particularly, protein sources, such as soy protein, can produce objectionable off-flavors in the finished products. For example, many consumers complain that high protein foods, like those supplemented with soy protein, taste grassy, beany, and bitter. Soy off-flavors may be responsible for most of the complaints with respect to the taste of soy-based products.

It is believed that the development of soy off-flavors is initiated when phospholipids and triglycerides undergo hydrolysis to yield polyunsaturated free fatty acids, which then react with molecular oxygen to form fatty acid hydroperoxides and other oxygenated lipid species. Both the hydrolysis and the oxidation can occur in enzyme-catalyzed and in non-enzyme-catalyzed reactions. The hydroperoxides then decompose into smaller molecules such as aldehydes and ketones and it is these small molecules that are responsible for the odor and flavor of vegetable oil-based products. Most of these flavor active molecules are derived from oxidation of polyunsaturated lipids. The formation of these flavor molecules and their hydroperoxide precursors begins as soon as the bean is crushed and continues through the soy isolate manufacturing process. Traditional processing methods have not been completely successful in reducing the level of off-flavors and off-flavor precursors to an acceptable level in finished soy isolate or in foods to which it is added.

The conventional process for manufacturing soy protein isolate begins with the production of a full fat flake from the bean, which is substantially defatted with hexane. This process typically removes more than 80% of the acid hydrolysable lipids in the flake, as measured by AOAC Method 922.06, while leaving behind the majority of the phospholipids present. Soy protein is then extracted from the defatted flour with water and separated from the insoluble vegetable matter via centrifugation. The extracted protein is precipitated, washed, resuspended in water and spray dried as described, for example, in Hettiarachchny, et al., Soybeans: Chemistry, Technology, and Utilization, pp. 379-411, Chapman & Hall (1997), which is incorporated herein by reference in its entirety.

These processes are unsuccessful in producing a soy protein composition with an acceptable flavor because the hexane is inefficient at removing all of the phospholipids and triglycerides that contain polyunsaturated fatty acids. Low levels of these off-flavor precursors, and some of the enzymes which act on them, remain after the hexane extraction. These components continue to generate off-flavors during the removal of hexane from the extracted flake at elevated temperatures. The defatted flake which serves as the source of the soy isolate thus typically contains about 2.8% to 5.0% of lipid (by weight dry basis), which may be analyzed as acid hydrolysable fat, and about 1.0% phospholipids (by weight dry basis), which may be analyzed by conventional HPLC methods. It also contains appreciable quantities of the flavor-active volatiles that persist through the subsequent protein isolation steps to result in isolate with the familiar grassy, beany, and bitter flavors.

One approach to improve the flavor of soy protein isolate is to remove the off-flavor molecules by extracting them with supercritical solvent, such as supercritical CO₂, after the protein has been isolated from the flake. For example, P. Maheshwari, E. T. Ooi, and Z. L. Nikolov, J. Amer. Oil Chem. Soc., 72:1107 (1995) extracted soy isolate with supercritical CO₂, liquid CO₂, and a mixture of 95% supercritical CO₂/5% ethanol. Although the extracted isolates had a lower intensity beany odor and improved overall acceptability compared with the starting isolate, each still retained significant flavor scores for beany odor. Thus, for the same reason outlined above, it is probable that high concentrations of phospholipids and oxygenated lipid species remain in the extracted isolates and cause the residual beany flavor.

While the prior art has demonstrated that supercritical CO₂ extraction may have an impact on the intensity of soy beany flavors, processes used to date have not been entirely satisfactory because they leave behind significant quantities of off-flavor precursors. These precursors quickly regenerate the beany off-flavors which a majority of consumers find to be unacceptable.

Many of the molecules that contribute to the flavor of fresh soy isolate gradually increase in concentration as the isolate ages during storage. This phenomenon increases the intensity of the off-flavor and makes the isolate less and less acceptable to consumers as it ages. Any process which decreases the rate at which these molecules are formed will lead to an increase in the shelf life of the soy isolate.

As is evident from the foregoing, a need exists in the industry for a soy protein isolate having reduced bitter flavor, and processes of making such a soy protein isolate. Additionally, it would be advantageous if the bitter components and molecules could be removed without a significant loss of protein from the soy protein isolate.

### SUMMARY OF THE DISCLOSURE

The present disclosure provides processes for removing bitter components and molecules from conventional available hydrolyzed soy protein isolates utilizing extraction and separation processes. In one embodiment for producing a soy protein isolate with reduced bitter components, the process includes extracting the bitter components from a hydrolyzed soy protein isolate utilizing an aqueous alcohol wash to produce a supernatant and a spent soy protein isolate, wherein the spent soy protein isolate has reduced bitter components, and then further separating the extracted bitter components from the supernatant utilizing a fractionation step. The separated extract of the supernatant has reduced bitter components and can be added back with the spent soy protein isolate recovered in the first extraction to produce a soy protein isolate having reduced bitter components. The soy protein isolates produced by the process of the present disclosure allow for improved flavor when used in a number of food products.

As such, the present disclosure is directed to a process for removing bitter components from a hydrolyzed soy protein isolate. The process comprises providing a hydrolyzed soy protein isolate; dispersing the hydrolyzed soy protein isolate in an aqueous alcohol wash to produce a slurry; centrifuging the slurry to produce a supernatant and a spent soy protein isolate having reduced bitter components; separately drying the supernatant and the spent soy protein isolate; dispersing the dried supernatant in an aqueous alcohol solution to produce an aqueous alcohol dispersion; separating the aqueous alcohol dispersion to produce a separated extract having reduced bitter components; drying the separated extract; and adding the dried separated extract to the dried spent soy protein isolate.

The present disclosure is further directed to a soy protein isolate having reduced bitter components. The soy protein isolate being prepared by a process comprising: providing a hydrolyzed soy protein isolate; dispersing the hydrolyzed soy protein isolate in an aqueous alcohol wash to produce a slurry; centrifuging the slurry to produce a supernatant and a spent soy protein isolate, wherein the spent soy protein isolate has reduced bitter components; separately drying the supernatant and the spent soy protein isolate; dispersing the dried supernatant in an aqueous alcohol solution to produce an aqueous alcohol dispersion; separating the aqueous alcohol dispersion to produce a separated extract having reduced bitter components; drying the separated extract; and adding the dried separated extract to the dried spent soy protein isolate.

The present disclosure is further directed to food products, such as emulsified meat products and drink products, comprising the soy protein isolate having reduced bitter components. Specifically, in one embodiment, the present disclosure is directed to an emulsified meat product comprising a processed meat and the soy protein isolate having reduced bitter components. In another embodiment, the present disclosure is directed to a food product comprising the soy protein isolate having reduced bitter components, the food product being selected from power bars, soups, sauces, breads, baked goods, breakfast cereals, dairy-type products, and the like. In yet another embodiment, the present disclosure is directed to a drink product comprising the soy protein isolate having reduced bitter components, wherein the drink product is selected from soft drinks, juices, sports drinks, and soy milk.

Other features and advantages of this disclosure will be in part apparent and in part pointed out hereinafter.

### DETAILED DESCRIPTION OF THE PREFERRED EMBODIMENTS

The present disclosure is generally directed to processes of producing a soy protein isolate with reduced off-flavors, and specifically, reduced bitter flavors. In one particular embodiment, the process for making a soy protein isolate with reduced off-flavors, specifically reduced bitter flavor, includes an extraction of the bitter components in a hydrolyzed soy protein isolate with an aqueous alcohol wash, followed by a further separation of the bitter components from the extracted supernatant obtained during the extraction using a solid-phase extraction (SPE) technique. Once the bitter components have been removed from the extracted supernatant, the extracted supernatant can be added back to the spent soy protein isolate extracted during the first extraction of bitter components to produce a soy protein isolate containing at least 90% (by weight dry basis) protein and having reduced bitter flavors. Specifically, it has been discovered that the bitter components in a hydrolyzed soy protein isolate can be extracted from the non-bitter components after production of the hydrolyzed soy protein isolate utilizing room temperature aqueous alcohol wash extractions to produce soy protein isolates with reduced off-flavors. As used herein the terms “soy protein isolate” and “soy isolate,” used interchangeably, mean a soy protein material comprising at least 90% (by weight dry basis) soy protein.

In one embodiment, the process for producing the soy protein isolate having reduced bitter components comprises: (1) providing a hydrolyzed soy protein isolate; (2) dispersing the hydrolyzed soy protein isolate in an aqueous alcohol wash to produce a slurry; (3) centrifuging the slurry to produce a supernatant containing bitter components and a spent soy protein isolate; (4) separately drying the supernatant and the spent soy protein isolate; (5) dispersing the dried supernatant in an aqueous alcohol solution to produce an aqueous alcohol dispersion; (6) separating the aqueous alcohol dispersion to produce a separated extract having reduced bitter components; and (7) adding the dried separated extract to the dried spent soy protein isolate.

This process starts with a hydrolyzed soy protein isolate. Typically, a soy protein isolate is produced by processing a soy protein source, such as soy flakes, by an extraction process using an aqueous alkaline wash. Extraction processes for forming soy protein isolates are well known and disclosed, for example, in U.S. Pat. No. 6,313,273, issued to Thomas, et al. (Nov. 6, 2001) and U.S. Pat. No. 6,830,773, issued to Porter, et al. (Dec. 14, 2004).

One process suitable for preparing a soy protein isolate for use in the processes described herein includes cracking soybeans to remove the hull, rolling them into flakes with flaking machines, defatting the flakes with hexane or heptane, subjecting the flakes to an aqueous extraction process, suspending the extracted soy protein in a wash solution, and precipitating a soy protein isolate therefrom. Suitable flaking machines may consist of a pair of horizontal counter-rotating smooth steel rolls. The rolls are pressed one against the other by means of heavy springs or by controlled hydraulic systems. The soybeans are fed between the rolls and are flattened as the rolls rotate one against the other. The roll-to-roll pressure can be regulated to determine the average thickness of the flakes. The rolling process disrupts the oil cell, facilitating solvent extraction (i.e., hexane or heptane) of the oil. Specifically, flaking increases the contact surface between the oilseed tissues and the extractant, and reduces the distance that the extractant and the extract will have to travel in the extraction process as described herein below. Typical values for flake thickness are in the range of 0.2 to 0.35 millimeters.

The defatted soy flake material may then be put through an aqueous extraction process. Typically, the aqueous extraction process is an aqueous alkaline wash. The aqueous alkaline wash removes materials soluble therein, including a substantial portion of the isoflavones and carbohydrates. This produces a protein material that contains at least 90% protein by weight on a dry basis, but which is significantly reduced in isoflavone concentration.

Typically, the alkaline wash has a pH of from 8.5 to about 10. The extraction is generally conducted by contacting the defatted soy flakes with an aqueous solution containing a set amount of base, such as sodium hydroxide, potassium hydroxide, ammonium hydroxide, and/or calcium hydroxide, and allowing the pH to slowly decrease as the base is neutralized by substances extracted out of the solid soy flakes. The initial amount of base is typically chosen so that at the end of the extraction operation the extract has a desired pH value, e.g., a pH within the range of from 8.5 to about 9.5. Alternatively, the pH of the aqueous phase can be monitored (continuously or at periodic time intervals) during the extraction and base can be added as needed to maintain the pH at a desired value.

Desirably, the aqueous alkaline wash should be a food grade reagent. The defatted soy flake material should be contacted with sufficient wash solution to form a soy protein extract. The weight ratio of wash solution to defatted soy flake material may be from about 2:1 to about 20:1, and preferably is from about 5:1 to about 10:1. Preferably, the defatted soy flake material is agitated in the wash solution and then centrifuged for a period of time to facilitate removal of materials soluble in the wash solution from the soy flake material. The wash solution is recirculated through the extractor until the residual oil content in the soy flakes is reduced to the desired level. The above described aqueous alkaline wash extraction removes water soluble components of the soy protein-containing material, such as carbohydrates and fat.

Once the soy protein has been extracted, it is suspended in a wash solution. Typically, the wash solution comprises water having a temperature of from about 90° F. to about 100° F. (32-38° C.). In a suitable embodiment, the extracted soy protein is suspended for 10 minutes at a temperature of 96° F. (35.6° C.). This water wash suspension further removes water soluble components of the extracted soy protein.

Finally, the suspended soy protein is precipitated with an acid to form a soy protein isolate. Precipitation separates remaining impurities, such as carbohydrates and fats, from the suspended soy protein. In one embodiment, to allow for sufficient precipitation, the acid is contacted with the suspended soy protein for a time period of about 5 minutes. Typically, the precipitation of the suspended soy protein is done at or near the isoelectric point of the soy proteins; that is, precipitation at a pH of from about 4.0 to about 5.0, preferably about 4.5. Suitable acids for precipitation can include, for example, hydrochloric acid, citric acid, phosphoric acid, and other organic and inorganic acids.

The above extraction, suspension, and precipitation steps can optionally be repeated one or more times to further remove impurities, such as carbohydrates and fat, from the soy protein isolate.

As noted above, the soy protein isolate to be used in the processes of the present disclosure is a hydrolyzed soy protein isolate. The soy protein isolate can be hydrolyzed using any means known in the art. For example, the soy protein isolate can be hydrolyzed using an enzyme treatment, heat treatment, or acid/alkali treatment during processing of the soy protein isolate. Particularly preferred for hydrolysis of the soy protein isolate for use in the present disclosure is an enzyme treatment.

Generally, the process for the enzyme hydrolysis of the soy protein isolate comprises diluting the soy protein isolate with water to form a soy protein slurry and adjusting the pH of the soy protein slurry to an alkaline pH with a suitable base. This is followed by heat-treating the pH-adjusted soy protein slurry and reacting the pH-adjusted soy protein slurry with an enzyme without maintaining the pH level to form an enzyme hydrolyzed soy protein mixture. The resulting enzyme hydrolyzed soy protein mixture is the soy protein isolate. Additional optional steps are described in more detail below.

In the first step described above, the soy protein isolate is diluted with water to form a soy protein slurry. Suitably, the soy protein isolate is diluted with water to produce a soy protein slurry that is about 8% to about 18% solids, by weight. Still more suitably, the soy protein slurry is about 10% to about 16% solids, by weight, and even more suitably, from about 12% to about 14% solids, by weight.

The pH of the soy protein slurry is then adjusted to a pH of from about 9.5 to about 10.5 with a suitable base. More suitably, the pH of the soy protein slurry is adjusted to about 9.8 to about 10.2 and even more suitably, to about 10.0. Suitable bases include sodium hydroxide, potassium hydroxide, and mixtures thereof. Preferably, the pH of the soy protein slurry is adjusted with sodium hydroxide.

The pH-adjusted soy protein slurry is then heat-treated. Preferably, the pH-adjusted soy protein slurry is heat-treated at a temperature and for a period of time to effectively denature the soy protein material contained in the soy protein slurry. Denaturation causes the soy protein material to unfold so that more of the insoluble soy protein material will be exposed to enzymatic hydrolysis upon addition of an enzyme to the soy protein slurry. Suitably, the pH-adjusted soy protein slurry is heat-treated at a temperature of from about 48° C. (118.4° F.) to about 58° C. (136.4° F.) for a period of time sufficient to denature the soy protein material. Still more suitably, the pH-adjusted soy protein slurry is heat-treated at a temperature of from about 48° C. (118.4° F.) to about 55° C. (131° F.), and even more suitably, at a temperature of from about 51° C. (123.8° F.) to about 53° C. (127.4° F.). The length of heat-treatment is suitably from about 30 minute to about 70 minutes. More suitably, the length of heat-treatment is from about 35 minutes to about 65 minutes. Preferred heat-treatment methods include direct or indirect heating with steam.

After the soy protein material contained in the pH-adjusted soy protein slurry is denatured, an enzyme is added to the pH-adjusted soy protein slurry. The preferred enzyme is an alkaline protease, which is suitably added to the pH-adjusted soy protein slurry at a level of from about 0.3% to about 0.6% solids basis. The enzyme hydrolysis of the soy protein material at an alkaline pH facilitates two reactions in the pH-adjusted soy protein slurry. In one reaction, the long chain peptides of the intact soy protein material are broken down by peptide hydrolysis. The other reaction is a deamidation reaction between the amide groups (—NH₃) of glutamines and hydroxide groups in the alkaline solution.

Representative alkaline proteases suitable for use in the processes of the present disclosure include Alcalase® (available from Novo Nordisk A/S, Denmark); Alkaline Protease Concentrate (available from Valley Research, South Bend, Ind.); and Protex™ 6L (available from Genencor, Palo Alto, Calif.). Preferably, the enzyme is Alcalase®.

The time period required for effective enzyme hydrolysis of the soy protein material is typically from about 30 minutes to about 60 minutes. More suitably, enzyme hydrolysis is allowed to occur for about 30 minutes to about 50 minutes, and even more suitably, enzyme hydrolysis is allowed to occur for about 35 to about 45 minutes.

During the reaction of the alkaline protease enzyme with the soy protein slurry, the pH is not maintained at a particular level. Rather, it is allowed to fluctuate according to the pH of the alkaline protease enzyme and the chemical processes that occur during the hydrolysis of the soy protein material contained in the pH-adjusted soy protein slurry. Typically, the pH of the resulting enzyme hydrolyzed soy protein mixture will have moved from about 9.5-10.5 to about 8.0-9.0. After the time period necessary for enzyme hydrolysis is complete, however, the pH of the enzyme hydrolyzed soy protein mixture is adjusted to a pH of from about 7.2 to about 7.6 with a suitable acid. More suitably, the pH of the enzyme hydrolyzed soy protein mixture is adjusted to about 7.4 with a suitable acid. Suitable acids include hydrochloric acid, phosphoric acid, citric acid, and mixtures thereof.

Commercially available hydrolyzed soy protein isolates can be used in the processes of the present disclosure. One particularly suitable hydrolyzed soy protein isolate is FXP 950, an enzyme hydrolyzed soy protein isolate, commercially available from The Solae Company (St. Louis, Mo.).

The soy protein isolates for use in the processes of the present disclosure suitably comprise at least 90% (by weight dry basis) soy protein. More suitably, the soy protein isolate comprises at least 90% (by weight dry basis) to about 95% (by weight dry basis) soy protein. In addition to the soy protein in the soy protein isolate, the soy protein isolate (in dry basis) generally comprises less than 1.0% (by weight) carbohydrates, from about 0.2% (by weight) to about 1.0% (by weight) fat, and less than 5.0% (by weight) ash.

Once the soy protein isolate is provided, the soy protein isolate is dispersed in an aqueous alcohol wash to produce a slurry. This dispersing process is conducted at room temperature with constant stirring, such as with a 0.25″×1″ Teflon-coated stirbar on a magnetic stirrer, stirring at a speed of approximately 60 revolutions per minute (rpm). Under these conditions, bitter components are extracted from the soy protein isolate.

A suitable aqueous alcohol wash is an aqueous solution of lower aliphatic alcohols, such as, methanol, ethanol, and isopropyl alcohol. One particularly preferred alcohol is ethanol. Typically, the aqueous alcohol wash includes from about 65% (by volume) to less than 100% (by volume) alcohol. More suitably, the aqueous alcohol wash includes from about 65% (by volume) to about 90% (by volume) alcohol. The desired amount of alcohol in the aqueous alcohol wash will depend on the end use of the soy protein isolate. In general, while an aqueous alcohol wash containing 65% (by volume) alcohol will solubilize, and thus, remove more bitter components compared to a wash with more alcohol, the removal is at the expense of extracting out higher quantities of soy protein as well. As such, when supplementing food products needing a higher level of protein, it would be desirable to use an aqueous alcohol wash with greater than 65% (by volume) alcohol to remove the bitter components from the soy protein isolate.

The aqueous alcohol wash typically used in the processes of the present disclosure is a neutral pH wash solution, that is, a wash solution having a pH of less than 8.5 and more than about 6.0. More suitably, the aqueous alcohol wash has a pH of from about 6.5 to about 8.0, and even more suitably, a pH of about 7.6.

Typically, the soy protein isolate is dispersed in the aqueous alcohol wash in a weight ratio of soy protein isolate:aqueous alcohol wash of from about 1:1.5 to about 1:20, and more suitably, about 1:10. The soy protein isolate is dispersed in the aqueous alcohol wash for a period of from about 30 minutes to about 180 minutes. More suitably, the soy protein isolate is dispersed in the aqueous alcohol wash for a period of from about 60 minutes to about 180 minutes, more suitably, from about 90 minutes to about 180 minutes, and even more suitably, about 120 minutes.

Once the soy protein isolate has been sufficiently dispersed in the aqueous alcohol wash to produce a slurry, the slurry is centrifuged to separate a liquid supernatant containing the soluble bitter components and an insoluble spent soy protein isolate. Typically, the slurry is centrifuged at a speed of from about 10,000 rpm to about 20,000 rpm, and more suitably, at a speed of from about 15,000 rpm to about 17,000 rpm, for a period of from about 5 minutes to about 25 minutes, and more suitably for about 15 minutes.

Both the supernatant and the spent soy protein isolate are then independently dried. One preferred method of drying the supernatant and the spent soy protein isolate is by evaporation. Suitably, the supernatant and the spent soy protein isolate are evaporated using an evaporator such as a Genevac EZ2 Evaporator (commercially available from Genevac, Inc., Valley Cottage, N.Y.) at a temperature of below 30° C. (86° F.), more suitably, at a temperature below 15° C. (59° F.).

The dried supernatant is further processed to separate the bitter components from the other components, such as isoflavones, saponins, peptides, carbohydrates, fats, ash, and the like. In one embodiment, the bitter components are separated from the dried supernatant by fractionating the dried supernatant using a solid-phase extraction (SPE) technique. Generally, SPE is an extraction method using both a solid and liquid phase to isolate organic analytes. Typically, the SPE procedure uses a solid sorbent material, typically C18, which is packed into a cartridge or imbedded in a disk, to perform essentially the same function as the aqueous alcohol wash in the above described extraction, which was used to separate the supernatant and spent soy protein isolate from the soy protein isolate-containing slurry. More specifically, a sample to be analyzed is prepared by first dispersing the sample in an organic solvent and then passing the sample through the solid sorbent to extract out the analyte. By first dispersing the sample in an organic solvent such as an alcohol, proper solvation or activation of the solid sorbent is achieved to keep the sample from simply flowing past the hydrophobic solid phase without contacting the analytes in the sample with the sorbent. For example, in one embodiment, the dried supernatant is dispersed in an aqueous alcohol solution prior to being loaded into a SPE cartridge.

Suitably, the aqueous alcohol solution includes from about 20% (by volume) to less than 100% (by volume) alcohol. More suitably, the aqueous alcohol solution includes from about 20% (by volume) to about 80% (by volume) alcohol, and even more suitably from about 20% (by volume) to about 40% (by volume) alcohol. Suitable alcohols for use in the aqueous alcohol solution include lower aliphatic alcohols, such as, methanol, ethanol, and isopropyl alcohol. One particularly preferred alcohol is ethanol.

Typically, the dried supernatant is dispersed in the aqueous alcohol solution in a weight ratio of dried supernatant:aqueous alcohol wash of from about 1:2 to about 1:20, and more suitably, from about 1:5 to about 1:16, and even more suitably, from about 1:10. The dried supernatant is dispersed in the aqueous alcohol solution for a period of less than 10 minutes. More suitably, the dried supernatant is dispersed in the aqueous alcohol solution for a period of less than about 5 minutes.

Once the dried supernatant is sufficiently dispersed to produce an aqueous alcohol dispersion, the dispersion is loaded in a SPE cartridge and fractionated to produce a separated extract having reduced bitter components. Any type of SPE cartridge known in the art can be used to fractionate the aqueous alcohol dispersion. In one particularly preferred embodiment, fractionation of the aqueous alcohol dispersion is carried out in a Supelco® Discovery DSC-18, which is a C18 SPE cartridge commercially available from Supelco (Bellefonte, Pa.). Specifically, in one preferred embodiment, 1 gram of dried supernatant can be loaded onto a 10-gram C18 SPE cartridge and separated at room temperature (approximately 72° F. (23° C.)).

In addition to the steps above, the separated extract can then be dried and then added back to the dried spent soy protein isolate from the first extraction. As such, the bitter components can be removed without losing significant protein from the hydrolyzed soy protein isolate. In one embodiment, the separated extract is dried using evaporation as described above. Specifically, the separated extract is evaporated using an evaporator such as a Genevac EZ2 Evaporator (commercially available from Genevac, Inc., Valley Cottage, N.Y.) at a temperature of below 30° C. (86° F.), more suitably, at a temperature below 15° C. (59° F.).

As noted above, the processes of the present disclosure produce soy protein isolates having reduced bitter components. As such, when used in foods and food products, there is a less bitter taste as compared to foods and food products using conventional soy protein isolates.

In addition to having reduced off-flavor due to removing the bitter components to produce soy protein isolates with reduced bitter components, the soy protein isolates produced using the processes of the present description have a reduced viscosity. Lower viscosity soy protein isolates may be intended for use in liquid products (i.e., beverages); and additionally, in some embodiments, lower viscosity soy protein isolates may be desired for use in a meat product. For example, lower viscosity soy protein isolates allow for improved water holding capacity of the meat product comprising the isolate.

As used herein, the term “viscosity” means the apparent viscosity of an aqueous slurry or a solution as measured with a rotating spindle viscometer utilizing a large annulus, where a particularly preferred rotating spindly viscometer is a Brookfield viscometer. In another embodiment, the viscosity can be measured using a Rapid Visco Analyzer (RVA) viscometer.

Typically, the soy protein isolates produced according to the present disclosure have a viscosity (at a 5% (by weight) aqueous slurry at ambient temperature) of less than about 15 cPs, more suitably, less than about 10 cPs, and even more suitably, less than about 5 cPs. More suitably, the viscosity of the soy protein isolates ranges from about 3 cPs to about 4.75 cPs and, even more suitably, from about 3 cPs to about 4.5 cPs.

The soy protein isolates of the present disclosure can be used in many consumer products such as soy milk, dairy-type products, bottled fruit drinks, power bars, soups, sauces, meat analogs, breads, baked goods, and breakfast cereals. Since the soy protein isolate of the current disclosure has reduced amounts of off-flavors, the taste of the above food products will not have the grassy, beany, and bitter off-flavor taste of traditional soy protein isolates while still providing the quality protein of soy protein isolate.

For example, in one embodiment, an emulsified meat product can be treated with the soy protein isolates having reduced bitter components to improve the meat product's flavor. As used herein, the term “emulsified meat product” refers to processed meats, wherein their ingredients have been mixed and/or injected with a soy protein isolate having reduced bitter components, such as the soy protein isolate described herein above.

Processed meats that can be treated with the soy protein isolates produced using the processes of the present disclosure can include, for example, hot dogs, sausages, bologna, ground meats, minced meats, meat patties, and the like, and combinations thereof. In one embodiment, the processed meat to be treated with the soy protein isolates of the present disclosure is a hot dog. In this embodiment, once the bitter components of the soy protein isolate have been removed using the processes described herein, the soy protein isolate is mixed in along with the other ingredients of the hot dog such as pork, chicken, spices, etc. The mixture is filled into a cellulose casing and then steam cooked at a temperature of 82° C. (180° F.) to form the emulsified meat product.

Typically, the food products that can be treated with the soy protein isolates having reduced bitter components can comprise from about 0.5% (by weight) to about 4% (by weight) soy protein isolate. More suitably, the soy protein isolates can be present in the amount of from about 0.5% (by weight) to about 3% (by weight), and even more suitably, from about 1% (by weight) to about 3% (by weight).

The soy protein isolates having reduced bitter components may also be used in drink products including, for example, acidic drink products such as soft drinks, juices, and sports drinks. Typically, the soy protein isolates are present in the drink products in an amount of from about 0.5% (by weight) to about 10% (by weight), more suitably, in an amount of from about 1% (by weight) to about 3% (by weight). The drink products in which the soy protein isolates are incorporated typically contain from about 70% (by weight) to about 90% (by weight) water. The drink products typically also contain sugars (e.g., fructose and sucrose) in an amount of up to about 20% (by weight).

Additionally, the soy protein isolates of the present disclosure can be used in combination with other proteins to produce soy protein products having a reduced amount of off-flavor. In particular, the soy protein isolate of the current disclosure can be used with dairy milk proteins to produce a soy product composition having a reduced amount of off-flavor. Suitable dairy milk proteins can include, for example, skim milk powder, whole milk powder, casein, sodium caseinate, calcium caseinate, whey protein concentrate, and whey protein isolate. These soy product compositions can then suitably be used in dairy products such as soy milk.

The following examples are simply intended to further illustrate and explain the present disclosure. The disclosure, therefore, should not be limited to any of the details in these examples.

### EXAMPLE 1

In this example, soy protein isolates are put through an extraction to produce spent soy protein isolates and supernatants. The spent soy protein isolates have reduced bitter components and thus, reduced bitter flavors.

Ten grams of FXP 950 (an enzyme hydrolyzed soy protein isolate commercially available from The Solae Co., St. Louis, Mo.) are dispersed in 100 milliliters of an aqueous ethanol wash containing 65% (by volume) ethanol to produce a slurry. The hydrolyzed soy protein isolate is dispersed in the aqueous ethanol wash at room temperature (approximately 23° C. (73.4° F.)) for 120 minutes with constant stirring. The slurry is centrifuged in a laboratory centrifuge available as Sorvall RC 5B Plus centrifuge (commercially available from Thermo Electron Corporation, Asheville, N.C.), spinning at approximately 17,000 revolutions per minute (rpm) for about 15 minutes. Both the supernatant (Sample B) and the spent soy protein isolate (Sample C) recovered from the centrifuging are evaporated completely using a Genevac EZ2 Evaporator (commercially available from Genevac, Inc., Valley Cottage, N.Y.) at a temperature of below 30° C. (86° F.).

A second batch of ten grams of FXP 950 is processed using the method described above with the exception of dispersing the hydrolyzed soy protein isolate in an aqueous ethanol wash containing 90% (by volume) ethanol to produce a slurry. As with the first batch above, both the supernatant (Sample D) and the spent soy protein isolate (Sample E) recovered from the centrifuging are evaporated completely using a Genevac EZ2 Evaporator at a temperature of below 30° C. (86° F.).

Both the recovered supernatant samples (Samples B and D) and spent soy protein isolate samples (Samples C and E) are independently dissolved in 200 milliliters of water and tasted by a subjective sensory panel to determine the astringency and bitterness of the sample. “Astringency” refers to the mouth feel, specifically, the mouth drying effect of a sample and “bitterness” refers to the sensory taste of a sample. The samples are compared to a 5% (by weight) slurry of FXP 950, which serves as a control (Sample A). Specifically, astringency and bitterness are measured using a 5-point hedonic scale. Five trained panelists taste the samples and evaluate the astringency and bitterness of the samples. According to the 5-point hedonic scale, a score of 5 is extremely astringent or bitter and a score of 0 is not astringent or bitter at all. By definition, the control FXP 950 soy protein isolate slurry sample is a 3 on the hedonic scale. The results of the panel tasting are shown in Table 1:

TABLE 1

| | Amount of | | |
|---|---|---|---|
| | Recovered | | |
| Sample | Material (g) | Astringency | Bitterness |
| Control (A) | N/A | 3.0 | 3.0 |
| | | (by definition) | (by definition) |
| Supernatant (B) | 4.5* | 2.1 | 4.4 |
| Spent soy protein isolate | 5.8 | 2.1 | 1.1 |
| (C) | | | |
| Supernatant (D) | 2.2* | 2.3 | 4.0 |
| Spent soy protein isolate | 8.0 | 0.8 | 0.8 |
| (E) | | | |

N/A = Not applicable
*Note:
Due to gummy nature, supernatant samples were not dried completely in this experiment.

As shown in Table 1, both samples of spent soy protein isolate are less astringent as compared to the control and have a less bitter taste as compared to both the control sample and the supernatant samples. As such, it appears that the bitter components of the hydrolyzed soy protein isolate are effectively extracted from the spent soy protein isolate and are left behind in the supernatant.

Additionally, the viscosity of spent soy protein isolate (E) is then analyzed using a Brookfield viscometer as described above. Specifically, the viscosity (at a 5% (by weight) aqueous slurry at ambient temperature) is determined and compared to the viscosities (under the same conditions) of a control sample using an FXP 950 isolate and a control sample using a SUPRO® 500e isolate (commercially available from The Solae Co., St. Louis, Mo.), both samples of which have not undergone the above extraction steps. The results are shown in Table 2.

TABLE 2

| Sample | Viscosity (cPs) |
|---|---|
| Spent soy protein isolate (E) | 3.75 |
| FXP 950 control | 4.63 |
| SUPRO ® 500e control | 20.05 |

As shown in Table 2, the viscosity of the spent soy protein isolate is lower than conventional isolates prior to being put through the extraction process. As noted above, this is advantageous in certain embodiments, such as when the soy protein isolates will be used in beverages and other liquid products and in certain meat products. Specifically, lower viscosity soy protein isolates allow for improved water holding capacity of meat products comprising the isolate, which can lead to a longer shelf life.

### EXAMPLE 2

In this Example, hydrolyzed soy protein isolates are put through extraction processes using various concentrations of ethanol to evaluate the ability of various aqueous ethanol washes in extracting spent soy protein isolates having reduced bitter components.

Ten grams of FXP 950 (a hydrolyzed soy protein isolate commercially available from The Solae Co., St. Louis, Mo.) are dispersed in 100 milliliters of an aqueous ethanol wash containing 95% (by volume) ethanol to produce a slurry. The hydrolyzed soy protein isolate is dispersed in the aqueous ethanol wash at room temperature (approximately 23° C. (73.4° F.)) for 120 minutes with constant stirring. The slurry is centrifuged in a Sorvall RC 5B Plus centrifuge (commercially available from Thermo Electron Corporation, Asheville, N.C.), spinning at approximately 15,000 revolutions per minute (rpm) for about 15 minutes. Both the supernatant (Sample F) and the spent soy protein isolate (Sample G) recovered from the centrifuging are evaporated completely using a Genevac EZ2 Evaporator (commercially available from Genevac, Inc., Valley Cottage, N.Y.) at a temperature of below 30° C. (86° F.).

A second batch of ten grams of FXP 950 soy protein isolate is processed using the method described above with the exception of dispersing the soy protein isolate in an aqueous ethanol wash containing 100% (by volume) ethanol to produce a slurry. As with the first batch above, both the supernatant (Sample H) and the spent soy protein isolate (Sample I) recovered from the centrifuging are evaporated completely using a Genevac EZ2 Evaporator at a temperature of below 30° C. (86° F.).

After the extraction process, all samples (supernatant and spent soy protein isolate (Samples F, G, H, I) were weighed to determine the percent recovery of the original hydrolyzed soy protein isolate. The recovery amounts of the samples were then compared to the recovery of the samples (Samples B, C, D, and E) from Example 1. The results are shown in Table 3.

TABLE 3

| Sample | Recovery (%) |
|---|---|
| Supernatant (B) | 45.0* |
| Spent soy protein isolate (C) | 58.0 |
| Supernatant (D) | 22.0* |
| Spent soy protein isolate (E) | 80.0 |
| Supernatant (F) | 22.3* |
| Spent soy protein isolate (G) | 81.5 |
| Supernatant (H) | 4.3 |
| Spent soy protein isolate (I) | 95.3 |

*Due to gummy nature, supernatant samples were not dried completely in this experiment

As shown in the data of Table 3, extraction using a higher concentration of ethanol will recover more of the sample as compared to using less ethanol.

Both the recovered supernatant samples (Samples F and H) and spent soy protein isolate samples (Samples G and 1) are independently then dissolved in 200 milliliters of water and tasted by a subjective sensory panel to determine the astringency and bitterness of the sample. The samples are compared to the spent soy protein isolate samples (Samples C and E) and supernatant samples (Samples B and D) of Example 1 and a 5% (by weight) slurry of FXP 950, which serves as a control (Sample A). Specifically, astringency and bitterness are measured using the 5-point hedonic scale of Example 1. Five trained panelists taste the samples and evaluate the astringency and bitterness of the samples. The results of the panel tasting are shown in Table 4:

TABLE 4

| Sample | Astringency | Bitterness |
|---|---|---|
| Control (A) | 3.0 (by definition) | 3.0 (by definition) |
| Supernatant (B) | 2.1 | 4.4 |
| Spent soy protein isolate (C) | 2.1 | 1.1 |
| Supernatant (D) | 2.3 | 4.0 |
| Spent soy protein isolate (E) | 0.8 | 0.8 |
| Supernatant (F) | 1.4 | 1.7 |
| Spent soy protein isolate (G) | 2.0 | 1.5 |
| Supernatant (H) | 0.6 | 0.2 |
| Spent soy protein isolate (I) | 1.9 | 2.1 |

N/A = Not applicable

As shown in Table 4, the samples of spent soy protein isolate, in which the bitter components have been removed, are less astringent and less bitter as compared to the control. More specifically, Sample E, which was obtained using a 90% (by volume) ethanol extraction, provides for the most efficient extraction of the bitter components without losing a significant amount of protein. At concentrations higher than 90% (by volume), the efficiency of extracting the bitter components appears to decrease.

### EXAMPLE 3

In this example, hydrolyzed soy protein isolates are put through an extraction process of the present disclosure to produce spent soy protein isolates having reduced bitter components and supernatants. The supernatants are then put through a separation process to produce separated extracts having reduced bitter components, which can then be added back to the spent soy protein isolates to recover the protein from the original hydrolyzed soy protein isolates.

Twenty grams of FXP 950 are dispersed in 200 milliliters of an aqueous ethanol wash containing 65% (by volume) ethanol to produce a slurry. The hydrolyzed soy protein isolate is dispersed in the aqueous ethanol wash at room temperature (approximately 23° C. (73.4° F.)) for 120 minutes with constant stirring. The slurry is centrifuged in a Sorvall RC 5B Plus centrifuge (commercially available from Thermo Electron Corporation, Asheville, N.C.), spinning at approximately 15,000 revolutions per minute (rpm) for about 15 minutes. Both the supernatant and the spent soy protein isolate recovered from the centrifuging are evaporated completely using a Genevac EZ2 Evaporator (commercially available from Genevac, Inc., Valley Cottage, N.Y.) at a temperature of below 30° C. (86° F.).

One gram of dried supernatant is then dispersed in an aqueous ethanol solution containing 20% (by volume) ethanol in a weight ratio of sample:solution of 1 gram:1.6 milliliters to produce an aqueous ethanol dispersion. The aqueous ethanol dispersion is then loaded in a Supelco® DSC-18 10 gram/60 milliliter SPE cartridge (commercially available from Supelco (Bellefonte, Pa.)). A first separated extract sample (Sample B) is collected from the first 8 milliliters of eluant processed in the cartridge. A second separated extract sample (Sample C) is collected from the second 8 milliliters of eluant processed in the cartridge. Another 16 milliliters of aqueous ethanol solution containing 40% (by volume) ethanol is then added to the SPE cartridge and the separated extract (Sample D) is collected. Finally, 16 milliliters of aqueous ethanol solution containing 80% (by volume) ethanol is added to the SPE cartridge and the separated extract (Sample E) is collected. All four samples are dried completely using a Genevac EZ2 Evaporator at a temperature of below 30° C. (86° F.).

The above separation process is repeated five more times to obtain enough of the separated extracts for the sensory evaluation described below. Each independent separation uses 1 gram of the dried supernatant.

All four samples (Samples B-E) are independently dissolved in 85.5 milliliters of water and tasted by a subjective sensory panel to determine the astringency and bitterness of the sample as described in Example 1 above. The samples are compared to a 10% (by weight) slurry of supernatant recovered from extraction of FXP 950 in aqueous alcohol wash containing 65% (by volume) ethanol, which serves as a control (Sample A). The results of the panel tasting are shown in Table 5:

TABLE 5

| | Amount of Recovered | | |
|---|---|---|---|
| Sample | Material (g) | Astringency | Bitterness |
| Control (A) | N/A | 3.0 (by definition) | 3.0 |
| | | | (by definition) |
| Sample (B) | 4.15 | 0.8 | 0.3 |
| Sample (C) | 0.82 | 1.4 | 0.5 |
| Sample (D) | 0.77 | 1.5 | 2.9 |
| Sample (E) | 0.33 | 1.6 | 4.0 |

N/A = Not applicable

As shown in Table 5, all four samples of separated extract are less astringent as compared to the control. Additionally, Samples B and C, which have the bitter components removed, are less bitter tasting as compared to control Sample A. As such, Samples B and C can be added back to the dried spent soy protein isolates obtained in the first extraction to produce a soy protein isolate having reduced bitter components.

Samples D and E are found to be as bitter, or more bitter than the control. As such, the bitter components are shown to be efficiently separated from the protein and other non-bitter components (in Samples B and C) using the separation process of the present disclosure.

The present disclosure is not limited to the above embodiments and can be variously modified. The above description of preferred embodiments is intended only to acquaint others skilled in the art with the disclosure, its principles and its practical application so that others skilled in the art may adapt and apply the disclosure in its numerous forms, as may be best suited to the requirements of a particular use.

With reference to the use of the word(s) “comprise” or “comprises” or “comprising” in this entire specification (including the claims below), it is noted that unless the context requires otherwise, those words are used on the basis and clear understanding that they are to be interpreted inclusively, rather than exclusively, and that it is intended each of those words to be so interpreted in construing this entire specification.

What is claimed is:

1. A process for removing bitter components from a hydrolyzed soy protein isolate, the process comprising:
providing a hydrolyzed soy protein isolate;
dispersing the hydrolyzed soy protein isolate in an aqueous alcohol wash to produce a slurry;
centrifuging the slurry to produce a supernatant and a spent soy protein isolate; and
separately drying the supernatant and the spent soy protein isolate, wherein the dried spent soy protein isolate has reduced bitter components;
dispersing the dried supernatant in an aqueous alcohol solution to produce an aqueous alcohol dispersion;
separating the aqueous alcohol dispersion to produce a separated extract having reduced bitter components;
drying the separated extract; and
adding the dried separated extract to the dried spent soy protein isolate.

2. The process as set forth in claim 1 wherein the aqueous alcohol wash comprises from about 65% (by volume) to less than 100% (by volume) alcohol.

3. The process as set forth in claim 2 wherein the aqueous alcohol wash comprises an alcohol selected from the group consisting of methanol, ethanol, and isopropyl alcohol.

4. The process as set forth in claim 3 wherein the alcohol is ethanol.

5. The process as set forth in claim 1 wherein the aqueous alcohol wash has a pH of from about 6.5 to about 8.0.

6. The process as set forth in claim 1 wherein the hydrolyzed soy protein isolate is dispersed in the aqueous alcohol wash for a period of from about 30 minutes to about 180 minutes prior to centrifuging.

7. The process as set forth in claim 1 wherein the slurry is centrifuged at a speed of from about 10,000 rpm to about 20,000 rpm.

8. The process as set forth in claim 1 wherein the supernatant and spent soy protein isolate are each dried using an evaporation process at a temperature of less than 30° C.

9. The process as set forth in claim 1 wherein the hydrolyzed soy protein isolate is hydrolyzed using an enzyme treatment.

10. The process as set forth in claim 9 wherein the enzyme for the enzyme treatment is a protease enzyme.

11. The process as set forth in claim 1 wherein the aqueous alcohol solution is an aqueous ethanol solution, the aqueous ethanol solution comprising from about 20% (by volume) to about 80% (by volume) ethanol.

12. The process as set forth in claim 1 wherein the separated extract is dried using an evaporation process at a temperature of less than 30° C.

13. A soy protein isolate having reduced bitter components, the soy protein isolate being prepared by a process comprising:
providing a hydrolyzed soy protein isolate;
dispersing the hydrolyzed soy protein isolate in an aqueous alcohol wash to produce a slurry;
centrifuging the slurry to produce a supernatant and a spent soy protein isolate;
separately drying the supernatant and the spent soy protein isolate, wherein the dried spent soy protein isolate has reduced bitter components;
dispersing the dried supernatant in an aqueous alcohol solution to produce an aqueous alcohol dispersion;
separating the aqueous alcohol dispersion to produce a separated extract having reduced bitter components;
drying the separated extract; and
adding the separated extract to the dried spent soy protein isolate.

14. The soy protein isolate as set forth in claim 13 having a viscosity (at a 5% (by weight) slurry) of less than about 15 cPs.

15. The soy protein isolate as set forth in claim 13 wherein the aqueous alcohol wash comprises from about 65% (by volume) to less than 100% (by volume) alcohol.

16. The soy protein isolate as set forth in claim 15 wherein the aqueous alcohol wash comprises an alcohol selected from the group consisting of methanol, ethanol, and isopropyl alcohol.

17. The soy protein isolate as set forth in claim 13 wherein the hydrolyzed soy protein isolate is dispersed in the aqueous alcohol wash for a period of from about 30 minutes to about 180 minutes prior to centrifuging.

18. The soy protein isolate as set forth in claim 13 wherein the slurry is centrifuged at a speed of from about 10,000 rpm to about 20,000 rpm.

19. The soy protein isolate as set forth in claim 13 wherein the supernatant and spent soy protein isolate are each dried using an evaporation process at a temperature of less than 30° C.

20. The soy protein isolate as set forth in claim 13 wherein the hydrolyzed soy protein isolate is hydrolyzed using an enzyme treatment.

21. The soy protein isolate as set forth in claim 20 wherein the enzyme for the enzyme treatment is a protease enzyme.

22. The soy protein isolate as set forth in claim 13 wherein the aqueous alcohol solution is an aqueous ethanol solution, the aqueous ethanol solution comprising from about 20% (by volume) to about 80% (by volume) ethanol.

23. An emulsified meat product comprising a processed meat and the soy protein isolate of claim 13.

24. The emulsified meat product as set forth in claim 23 wherein the processed meat is selected from the group consisting of hot dogs, bologna, ground meats, minced meats, and combinations thereof.

25. A food product comprising the soy protein isolate of claim 13.

26. The food product as set forth in claim 25 selected from the group consisting of protein bars, soups, sauces, breads, baked goods, breakfast cereals, dairy-type products.

27. A drink product comprising the soy protein isolate of claim 13.

28. The drink product as set forth in claim 27 selected from the group consisting of soft drinks, juices, and sports drinks.

29. The drink product as set forth in claim 27 wherein the drink product is soy milk.
